(19) United States
(12) Patent Application Publication
(10) Pub. No.: US 2009/0071823 A1
(43) Pub. Date: Mar. 19, 2009

(54) Disposable enzymatic sensor for liquid samples

(75) Inventors: Michael Catt, Sharnbrook (GB); Barry Gerald Denis Haggett, Luton (GB); Aine Martina O'Connor, Sharnbrook (GB); Heather Elaine Roxborough, Fife (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/221,856

(22) Filed: Aug. 7, 2008

(30) Foreign Application Priority Data
Aug. 10, 2007 (EP) EP07114189

(51) Int. Cl. C12N 11/08

(52) U.S. Cl. 204/403.04

(57) ABSTRACT

The present invention relates to a disposable electrochemical sensor which can measure redox species in a liquid sample

The present invention provides a disposable electrochemical sensor which can accurately quantify redox species in a liquid sample. The inventive sensor comprises at least one specific binding agent such as an enzyme, anti bodies, nucleotide sequence etc.

Electrochemical biosensors are known from the prior art, such as U.S. Pat. No. 4,795,542 or WO03104793.

For quantitative analysis, it can be important to control and keep constant the pH of the sample volume in contact with the measurement electrodes. This may be a small or even trivial problem where the original sample is already strongly buffered to a pH appropriate for measurement of the analyte (e.g. the measurement of blood glucose using disposable sensors incorporating glucose oxidase). In other cases the original sample may be strongly buffered to a pH far from the optimum range for the analytical measurement, e.g. urine may be buffered at pH 6.5 whereas the determination of ascorbate may be optimally carried out at pH 2.9. In such cases, it can be a significant technical challenge to provide sufficient buffer to move the pH of the measured sample volume to the desired value in a reproducible manner which does not require intervention by the user. It is notable that much of the prior art uses examples whereby the sample is naturally buffered to an appropriate pH (e.g. blood) or the sample is provided by model solutions (which are not adversely buffered).

In WO03104793 a biosensor with a filter device is described. The biosensor described therein has an electrode, which can be covered by (or can contain) a mediator compound and wherein the filter can comprise a means for pH alteration.

These embodiments possess some disadvantages.

If the buffer system has to be added to the sample before or during the measurement, there are a few sources of error, which exist. The concentration of the system has to be exact, the amount of liquid has to be exact, etc.

Furthermore, the addition of the buffer is an additional step in the procedure.

The incorporation of the buffer system into the filter system as disclosed (only hypothetical, no examples of such a device are described) leads to various problems. For example the production of such filter systems has to be done in a reproducible manner and such systems are not described elsewhere. Furthermore if the buffer system is present in a liquid form, there is the problem of controlling dilution. When the pH buffer is present in a solid form there is a problem of solubilisation of the buffer as well as obtaining a homogenous sample solution.

Furthermore the addition of a buffer system in the filter device impedes sample flow, and leads to a poor distribution of the liquid sample and may result in a variation of pH which distorts the measurement.

The other major problem is wash-up—where the buffer in the filter simply washes past the measurement electrode structure and so the pH of the sample fluid is not adjusted correctly at the site of measurement.

Buffer may also be incorporated into the interfacial layer—but it is difficult to incorporate a large quantity of buffer components into this layer in a reproducible manner. Either or both of the buffer components are inclined to precipitate from the mixture and/or form two phase systems which require adverse amounts of surfactant and other additives potentially detrimental to subsequent manufacture, storage or use of the sensors. For example, metaphosphoric acid (which is usual supplied as a mixture with sodium phosphite—and is thus a good buffer) when mixed with polyethylene glycols leads to the formation of two phase aqueous solutions at quite small levels of acid loading.

Therefore there is still a need for an improved disposable sensor for quantifying liquid samples.

Surprisingly it has been found out that it is possible to achieve a substantial loading of buffer components (at least 20 weight-%) in the interfacial layer when at least one binder and at least one buffer component are used. This results in an interfacial layer which is also suitable for an easy manufacture, storage and use.

The sensor system according to the present invention allows a high loading of buffer components in the interfacial layer without precipitation, formation of two-phase aqueous systems, or other cause that hinders manufacture, storage or use of the sensor.

Due to the use of a binder as defined below the buffer system is evenly distributed.

Therefore the present invention relates to a disposable electrochemical sensor for quantifying one or more redox species in a liquid sample, comprising

an electrode system, and
an insulating layer, and
a filter system, and
an interfacial layer, and
at least one specific binding agent,
characterized in that the interfacial layer comprises at least one binder and at least one buffer system.

The disposable electrochemical sensor according to the present invention does comprise specific binding agent, such as enzymes (as to be found in U.S. Pat. No. 4,795,542, GB-A-1 554 292, US-A-5 746 898 and WO-A-93/13408) or antibodies or any other commonly known and used specific binding agent. Antibodies are another subclass of proteins. Each antibody molecule is made up of four peptide chains joined by disulfide bonds into a generally Y-shaped molecule. Antibodies, also called immunoglobulins, are produced by B cells as a primary immune defence. Each antibody has a unique binding site that can combine with a complementary site of a foreign antigen.

Suitable enzymes are glucose oxidase, galactose oxidase, lactate oxidase, alcohol oxidase, cholesterol oxidase, uricase, ascorbate oxidase, pyruvate oxidase, hexokinase, catalase, urease, creatine deiminase, glutamate oxidase, lysine oxidase, leucine hydrogenase, lactate dehydrogenase, sarcosine oxidase and creatine amidohydrolase. The specific biding agent can be incorporated anywhere in the disposable electrochemical sensor. Usually it is incorporated into the electrode system and/or the interfacial layer. It is also possible that the specific binding agents form a separate layer in the disposable electrochemical sensor.

Usually the concentration of a specific binding agent in a disposable electrochemical sensor according to the present invention is between 0.001 U/cm² and 100 U/cm² (units per cm²).

In addition it is also possible and useful to incorporate buffer system into the filter means.

In a preferred disposable electrochemical sensor according to the present invention, the interfacial layer comprises from 20 to 50 weight-% (wt-%) (dry weight) of at least one buffer system, based on the total dry weight of the interfacial layer.

A disposable electrochemical sensor according to the present invention is usually used in conjunction with a separate device that is capable of measuring the electrochemical signals generated by the sensor according to the present invention.

Usually a disposable electrochemical sensor according to the present invention is loaded with the liquid sample and then put into the device, which measures the signal and usually shows the result to user. But it also possible to provide a disposable electrochemical sensor according to the present invention to measure the signal itself.

A disposable electrochemical sensor according to the present invention can have any suitable geometrical form. Usually it is in the form a rectangle. The size of the rectangle can vary, but preferred is a sensor which is easy to handle, that means not too small but also not too large. Usually it is has a width of 0.5 to 3 cm and a length of 3 to 10 cm.

The disposable electrochemical sensor according to the present invention has usually a thickness of 0.2-2 millimetres.

The expression “disposable” is used herein to indicate that each sensor has the capacity for only a single measurement.

For the purpose of the present invention a “redox species” refers to an electrochemically active analyte comprising a moiety capable of electrochemical oxidation and/or reduction at an electrode (wherein oxidation gives a positive current and reduction gives a negative current) within the operating range of standard, accumulation and stripping electrochemical techniques. Preferably such electrochemical techniques are within the operating window of +/−2 volts.

The liquid sample can have any origin. It can be a biological sample, microbiological fluid, beverage, food suspension, etc.

The disposable electrochemical sensor according to the present invention is very suitable for testing liquid samples comprising components having a biological origin, preferably a biological liquid sample will comprise plant or animal derived materials in the form of a plant or animal extract. Most preferably a biological liquid sample will comprise one or more components of human origin derived from or comprising fluids selected from the group comprising sweat, saliva, blood, tears and urine.

A disposable electrochemical sensor according to the present invention comprises an electrode system.

The electrode system preferably comprises a working electrode, a counter electrode and a reference electrode, however it is recognised that the reference and counter electrodes may be combined in some circumstances, e.g. when the measurement of current is small (˜pA).

The working electrode acts as a source or sink of electrons for exchange with molecules in the interfacial region (the solution adjacent to the electrode surface), and must be an electronic conductor. It must also be electrochemically inert (i.e., does not generate a significant current in response to an applied potential) over a wide potential range (the potential window). Commonly used working electrode materials for cyclic voltammetry include platinum, gold, mercury, dried carbon ink and glassy carbon. Other materials (e.g., semiconductors and other metals) are also used, for more specific applications. The choice of material depends upon the potential window required (e.g., mercury can only be used for negative potentials, due to oxidation of mercury at more positive potentials), as well as the rate of electron transfer (slow electron transfer kinetics can affect the reversibility of redox behaviour of the system under study). The rate of electron transfer can vary considerably from one material to another, even for the same analyte, due to, for example, catalytic interactions between the analyte and active species on the electrode surface.

During any electrochemical experiment, a redox reaction occurs at the surface of the counter electrode (to balance the redox reaction at the surface of the working electrode), and the products of this reaction can diffuse to the working electrode and interfere with the redox reaction occurring at that site. However, in electroanalytical measurements such as cyclic voltammetry, the time scale of the measurement is too short for this diffusion to be able to cause significant interference, so there is no need to place the auxiliary electrode in a separate compartment.

A reference electrode is an electrode which has a stable and well-known electrode potential. The high stability of the electrode potential is usually reached by employing a redox system with constant (buffered or saturated) concentrations of each participant in the redox reaction. Reference electrodes are used to measure electrochemical potential. A suitable and commonly used reference electrode is a Ag/AgCl electrode.

The major requirement for a reference electrode is that the potential does not change significantly during the measurement period. Since the passage of current through an electrode can alter the potential, such effects are minimized for the reference electrode in the three electrode system by a) having a high input impedance for the reference electrode (thereby decreasing the current passing through the reference electrode to negligible levels) and b) using a non-polarizable electrode as the reference electrode (i.e., the passage of small currents does not alter the potential).

The working and counter electrodes which are made from dried carbon ink are particularly effective for the quantification of ascorbate in a liquid sample. A suitable carbon ink for this purpose is D14 available from Gwent Electronic Materials, Pontypool, Gwent.

Measurement electrode systems for construction of a disposable electrochemical sensor according to the present invention preferably comprise noble metals, most preferably the electrode comprises one or more elements selected from the group comprising gold, platinum, rhodium, palladium, rhenium but carbon will suffice in many applications including where the sample is ascorbate.

The counter electrode should be of sufficient size in relation to the working electrode such that the electrochemical reaction at the charge transfer interface is not limited. Preferably the counter electrode is at least 5 times the size of the working electrode, more preferably at least 10 times the size.

The disposable electrochemical sensor according to the present invention further comprises an insulating layer placed over the electrode system so as to expose only those parts necessary to make contact with the measurement sample and those parts necessary to make contact with the potentiostat or other measuring equipment. One such suitable insulating material is the dielectric (D2040917D2) available from Gwent Electronic Materials, Pontypool, Gwent.

The disposable electrochemical sensor according to the present invention further comprises a filter mean, which comprise at least one compound usually chosen from the group consisting of synthetic membranes, nitrocellulose, cellulose, silica, glass fibres, filter paper, agar gel and other materials known in the art for chromatic binding of proteins.

Furthermore the disposable electrochemical sensor according to the present invention always comprises an insulating layer. Such a layer can be built up by commonly known (dielectric) material used for that purpose. Suitable materials are e.g. insulating resin made from vinylpolyester(s), polyimide(s), polyethylene terephthalate, etc.

Furthermore the disposable electrochemical sensor according to the present invention comprises always an interfacial layer.

The interfacial layer is usually between the filter means and the electrode system, wherein said interfacial layer has a lower energy requirement to transfer the liquid sample to electrode surface than the filter material. In other words, there is a lower energy of wetting between the interfacial layer and the electrode system than there would be if there were direct contact between the filter means and the electrodes.

The interfacial layer may be regarded as a matrix capable of forming a transfer layer when wetted by the liquid sample and thus the interfacial layer can be provided dry before use, i.e. to be wetted by the liquid sample which contains the redox species to be detected.

The interfacial layer can also comprises further components, such as for example filler material (e.g. silicas), to give the mixture desirable attributes such appropriate viscosity, stability, etc.

The interfacial layer of the disposable electrochemical sensor according to the present invention comprises always at least one binder material, which is a polymeric material. Such a polymeric material has a preferred molecular weight of 600 to 10000, more preferred 5000 to 8000. A very preferred polymeric material has a molecular weight of about 6000. The molecular weight can be determined according to well known processes.

Preferred polymeric materials are polyvinylpyrrolidone (PVP), polyethylenoxide (PEO), polyethyleneglycol (PEG), starch or gelatine.

Very preferred is PEG with the molecular weight as described above.

The amount of the incorporated buffer system can vary. It is dependent for example on the use of the sensor as well as on the kind of the buffer system.

Usually an interfacial layer of a disposable electrochemical sensor according to the present invention can comprise 20 to 50 wt-% (dry weight) of a buffer system, based on the total dry weight of the interfacial layer.

As further embodiment of the present invention buffer system can also be incorporated into the filter mean. Typically, the amount can go from 0 to 20 mg cm⁻² depending on the type of filter material.

The buffer system can be incorporated in liquid form as well as in solid (dry) form. The incorporation of the buffer into the interfacial layer as well as the filter can be done by commonly known methods.

Any commonly known pH buffer system can be used for the present invention. Usually the pH is buffered at a level between 1.5 and 9.

Depending on the sample, a suitable buffer system can be chosen.

Suitable pH buffers are

alkaline buffers systems, such as
N,N-(bis-2-hydroxymethyl)glycine (BICINE)-NaOH buffer (pH 7.9-8.9)
borate buffer: Na₂B₄0₇.10H₂O, 0.1M HCl (pH 8.1-9)
N-(tris(hydroxymethyl)methyl) glycine (TRICINE)-NaOH (pH 7.4-8.6)
N-2-hydroxyethylpiperazine-N′-3-propanesulphonic acid (EPPS)-NaOH (pH 7.5-8.7).
Acid buffers systems such as
sodium acetate, acetic acid (pH 3.7-5.6)=acetate buffer
sodium succinate, succinic acid (pH 3.8-6.0)=succinate buffer
dimethylkglutaric acid-NaOH buffer (pH 3.2-7.6)
sodium citrate-citric acid (pH 3-6.2)=citrate buffer
McIlvaine buffer (citrate-phosphate) (pH 2.6-7.6)

For example when ascorbate is measured the pH preferably should be below 7. The preferred pH range is 2.5 to 3.5, and the optimum pH is around 2.9.

Alternatively a stronger acid such as metaphosphoric acid may be used to lower the pH of the solution below pH 3.5. Where a mediator such as ferricyanide is employed, care must be taken not to lower the pH to a level where the mediator breaks down.

The thickness of the interfacial layer is usually between 2 μm to 200 μm.

Furthermore the disposable electrochemical sensor according to the present invention can also comprise at least one redox mediator. The redox mediator can be part of the interfacial layer and/or of the filter. It can also create a layer on its own.

The redox mediator can be organic, inorganic, coordination compounds with inorganic or organic ligands, as well as organometallic compounds.

Suitable redox mediators are quinones, ruthenium bipyridyl complexes, ferrocyanide, ferricyanide, ferrocene, ferrocene carboxylic acid and cobalt phthalocyanine according to the redox reaction properties.

The layers as described above are put onto a suitable substrate, which is the basis for the disposable electrochemical sensor. The choice of this material is not critical. Usually it is a polymeric substrate such as PVC, PE, PP, etc.

The disposable electrochemical sensor according the present invention can be produced by commonly known used methods.

The disposable electrochemical sensor according to the present invention is used to measure quantitatively, semi-quantitatively or qualitatively the amount of the redox species.

Redox species for the purpose of the present invention are preferably selected from the group of vitamins comprising ascorbate (vitamin C), vitamin E; antioxidant nutrients selected from the group comprising reduced glutathione, polyphenols, catechols, flavones such a quercetin, isoflavones such as phytoestrogens; heteroaromatic compounds such as penicillin, aspirin, carbazole, murranes; aromatics such as phenols, carbonyls and benzoates; trace metal ions selected from the group comprising nickel, copper, cadmium, iron and mercury.

In a most preferred embodiment the redox species is ascorbate as this is a good indicator of human nutrition.

Measurement for the purpose of quantification is suitably performed by a potentiostat. This may comprise a device into which the sensor as described above is inserted for a reading to be taken or more alternatively may be built into the disposable device provided to the consumer.

### DESCRIPTION OF THE FIGURES

FIG. 1: systematic description of a sensor according to the present invention:

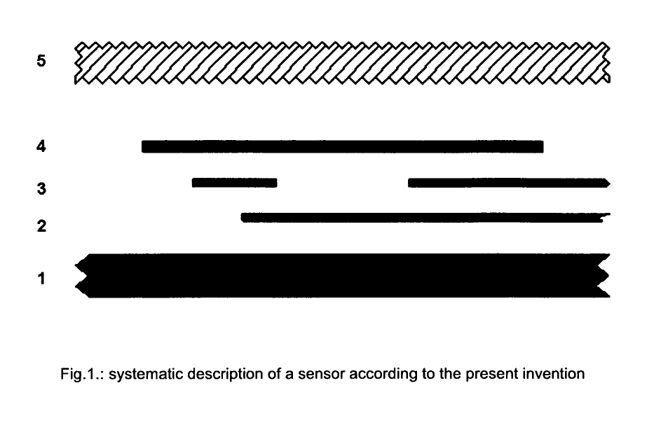

1 is the basis substrate,
2 is the electrode system (the working electrode is covered by the specific binding agent)
3 is the insulating layer
4 interfacial layer (comprising the specific binding agent, the binder and the buffer system)
5 is the filter means (e.g. glass fibre)

The following example serves to illustrate the invention:

### EXAMPLE 1

A disposable electrochemical sensor consists of
(i) a glass fibre filter means (Whatman GF/A, thickness ˜290 μm at 53 kPa);
(ii) interfacial layer consisting of PEG (6000 D), redox mediator and citrate buffer—the composition of the layer is given in Table 1;
(iii) insulating layer printed using a dielectric material;
(iv) electrode system, which is made from carbon ink—overprinted with Ag/AgCl where necessary to form a reference electrode element; the working electrode is covered by 0.003 u/cm2 of ascorbate oxidase; and
(v) a base substrate (polyester).

The electrode system is printed onto the base substrate, the insulating layer is printed over the electrode system, as required, and the interfacial layer is coated onto it. Afterwards the glass fibre layer is fixed onto the mediator layer by an adhesive.

The mediator and buffer components of composition described in Table 1 can be screen printed or paste deposited in accordance with the FIGURE.

TABLE 1
Composition of the interfacial layer

| Components | % (w/w) |
|---|---|
| PEG 6000 | 30.00 |
| Citric acid monohydrate | 14.48 |
| Trisodium citrate dihydrate | 8.27 |
| Potassium chloride | 0.39 |
| Potassium ferricyanide | 1.88 |
| D.I. Water | 34.98 |
| Silica gel Davis grade 633 | 10.00 |

1. A disposable electrochemical sensor for quantifying one or more redox species in a liquid sample, comprising
an electrode system, and
an insulating layer, and
a filter system, and
an interfacial layer, and
at least one specific binding agent,
characterized in that the interfacial layer comprises at least one binder and at least one buffer system.

2. A disposable electrochemical sensor according to claim 1 wherein a buffer system is incorporated into the filter system.

3. A disposable electrochemical sensor system according to claim 1 wherein the interfacial layer comprises from 20 to 50 wt % (dry weight) of at least one buffer system, based on the total dry weight of the interfacial layer.

4. A disposable electrochemical sensor according to claim 1, wherein the sample is a liquid biological sample, microbiological fluid, beverage or food suspension.

5. A disposable electrochemical sensor according to claim 1, wherein the sample is sweat, saliva, blood, tears and urine.

6. A disposable electrochemical sensor according to claim 1, wherein the sensor comprises
(i) an electrode system, and
(ii) optionally a reference electrode.

7. A disposable electrochemical sensor according to claim 5, wherein the electrode system comprises
(i) a working electrode,
(ii) a counter electrode and
(iii) a reference electrode.

8. A disposable electrochemical sensor according to claim 6, wherein the reference electrode is an Ag/AgCl electrode.

9. A disposable electrochemical sensor according to claim 7, wherein the working and counter electrodes are made from dried carbon ink.

10. A disposable electrochemical sensor according to claim 7, wherein the counter electrode is at least 5 times the size of the working electrode, preferably at least 10 times the size.

11. A disposable electrochemical sensor according to claim 1 wherein the binder is a polymeric material having a molecular weight of 600 to 10000.

12. A disposable electrochemical sensor according to claim 1 wherein the binder is PEG.

13. A disposable electrochemical sensor according to claim 1, comprising at least one redox mediator.

14. A disposable electrochemical sensor according to claim 13, wherein the redox mediator is chosen from the group consisting of quinones, ruthenium bipyridyl complexes, ferrocyanide, ferricyanide, ferrocene, ferrocene carboxylic acid and cobalt phthalocyanine.

15. A sensor according to claim 1 wherein the buffer system is chosen from the group consisting of alkaline buffers systems, such as N,N-(bis-2-hydroxymethyl)glycine-NaOH buffer; borate buffer, N-(9-trishydroxymethyl)methyl)glycine-NaOH; N-2-hydroxyethylpiperazine-N′-3-propanesulphonic acid (EPPS)-NaOH; acid buffers systems, such as acetate buffer, succinate buffer, dimethylkglutaric acid-NaOH, citrate buffer, McIlvaine buffer or metaphosphoric acid.

16. A sensor according to claim 1 wherein, wherein the specific binding agents can be chosen from the group consisting of glucose oxidase, galactose oxidase, lactate oxidase, alcohol oxidase, cholesterol oxidase, uricase, ascorbate oxidase, pyruvate oxidase, hexokinase, catalase, urease, creatine deiminase, glutamate oxidase, lysine oxidase, leucine hydrogenase, lactate dehydrogenase, sarcosine oxidase and creatine amidohydrolase.

17. Use of a sensor according to claim 1 for quantifying one or more redox species in a liquid sample.
